SEQUENCE LISTING (1 sequence)

SEQ ID NO: 1 - protein, 282 aa, unidentified
  source 1..282: note = amino acid sequence of antigen protein vinculin
PKFREAVKAASDELSKTISPMVMDAKAVAGNISDPGLQKSFLDSGYRILGAVAKVREAFQPQEPDFPPPPQLRLTDELAPPKPPLPEGEVPPPRPPPPEEKDEEFPEQKAGEVINQPMMMAARQLHDEARKWSSKGNDIIAAAKRMALLMAEMSRLVRGGSGTKRALIQCAKDIAKASDEVTRLAKEVAKQCTDKRLLQVCERIPTISTQLKILSTVKATMLGRTNISDEESEQATEMLVHNAQNLMQSVKETVREAEAASIKIRTDAGFTLRWVRKTPWYQ

(19) United States
(12) Patent Application Publication
(10) Pub. No.: US 2023/0333097 A1
(43) Pub. Date: Oct. 19, 2023

(54) KIT FOR DETECTING ANTI-VINCULIN-IMMUNOGLOBULIN G (IgG) ANTIBODY

(71) Applicant: ZHEJIANG UNIVERSITY, Hangzhou City (CN)

(72) Inventors: Qing YE, Hangzhou City (CN); Jianhua MAO, Hangzhou City (CN); Dandan TIAN, Hangzhou City (CN)

(73) Assignee: ZHEJIANG UNIVERSITY, Hangzhou City (CN)

(21) Appl. No.: 17/855,993

(22) Filed: Jul. 1, 2022

(30) Foreign Application Priority Data
Jul. 1, 2021 (CN) 202110743524.X

(51) Int. Cl. G01N 33/543; G01N 33/544; G01N 33/68

(52) U.S. Cl. CPC G01N 33/54306; G01N 33/54326; G01N 33/544; G01N 33/6893; G01N 33/6854; G01N 2333/4701; G01N 2800/347

(57) ABSTRACT

The present disclosure provides a kit for detecting an anti-vinculin-immunoglobulin G (IgG) antibody, including the antigen protein vinculin, a solid phase carrier, a labeled antibody, an antigen diluent, a sample dilution buffer, an antibody diluent, a substrate color development reagent, a washing solution, a standard, a positive quality control, and negative quality control. In the present disclosure, the kit can detect the anti-Vinculin-IgG antibody in a sample to be tested by indirect reaction combined with magnetic particle-based chemiluminescence immunoassay. Autoantibodies against the target antigen vinculin are identified in the serum of a patient with autoimmune nephrotic syndrome for the first time, and a detection kit is provided for the autoantibodies. The present disclosure provides a basis for molecular mechanism research and clinical diagnosis and treatment of autoimmune nephrotic syndrome related to vinculin and vinculin-IgG autoantibodies at home and abroad.

### CROSS-REFERENCE TO RELATED APPLICATION

This application claims the benefit and priority of Chinese Patent Application No. 202110743524.X, filed on Jul. 1, 2021, the entire disclosure of which is hereby expressly incorporated by reference.

### TECHNICAL FIELD

The present disclosure belongs to the technical field of biomedicine and relates to a kit for detecting an anti-vinculin-immunoglobulin G (IgG) antibody.

### REFERENCE TO AN ELECTRONIC SEQUENCE LISTING

The contents of the electronic sequence listing (GWP2022060219.xml; Size: 2,204 bytes; and Date of Creation: Aug. 22, 2022) is herein incorporated by reference in its entirety.

### BACKGROUND ART

In recent years, there have been an increasing number of kidney diseases in children, among which autoimmune nephrotic syndrome has the highest incidence rate, seriously endangering children's physical and mental health. Autoimmune nephrotic syndrome is a type of clinical syndrome caused by increased permeability of a glomerular filtration membrane, leading to increased plasma protein filtration to generate massive proteinuria. Autoimmune nephrotic syndrome may bring symptoms to patients, mainly manifested as massive proteinuria, hypoalbuminemia, and severe edema. Ali et al. observed that after transplantation of kidneys from patients with refractory minimal change nephrotic syndrome (MCNS), the recipients had normal renal function without any proteinuria. The cause of MCNS is not all in the kidney itself but may be mainly due to the patient's internal environment. In addition, except for some pediatric patients with genetic defects, most pediatric patients with autoimmune nephrotic syndrome can improve after treatment with hormones and immunosuppressants, indirectly proving that the syndrome is closely related to the patient's autoimmunity.

For the past few years, it has been found that B-cell dysfunction also plays a vital role in autoimmune nephrotic syndrome. In recent years, multiple multicenter clinical studies worldwide have shown that rituximab (RTX) can be successfully used for treating MCNS, especially for treating refractory nephrotic syndromes with a desirable therapeutic effect. However, studies have also found that during the treatment of hormone-dependent nephrotic syndromes with RTX, the effect of RTX in removing B cells can be maintained for approximately five months. From 6 to 7 months, the patient's condition may also relapse as the number of B cells recovers. This suggests pathological B-cell clones exist in patients with autoimmune nephrotic syndrome. Identifying and precisely removing these pathological B-cell clones is beneficial to the recovery of autoimmune nephrotic syndrome. It reduces the risk of humoral immune deficiency in patients due to indiscriminate B-cell clearance by means such as RTX. However, to date, the target antigens targeted by pathological B cells remain unclear in pediatric patients with autoimmune nephrotic syndrome. Pathologically, MCNS or focal segmental glomerulosclerosis9 is considered a podocyte disease with massive proteinuria due to loss or alteration of podocyte function. Podocytes are glomerular epithelial cells in the kidney that attach to the outside of a basement membrane of the glomerulus and are the last guarantee against protein loss. Podocyte damage generally causes massive proteinuria.

Vinculin is a cytoplasmic protein that binds to actin and is involved in cell adhesion. One study found that a self-antibody titer of an anti-vinculin antibody is inversely proportional to the density of Cajal (ICC) interstitial cells in the gastric muscular plexus of a gastric cancer patient (Kim J H, Nam S J, Park S C, et al. Association between interstitial cells of Cajal and anti-vinculin antibody in the human stomach. Korean J. Physiol. Pharmacol, 24:185-191.). Vinculin has also been studied in autoimmune diseases. Brittany L. Adler et al. found that compared with healthy controls, patients with systemic sclerosis have a higher level of anti-vinculin autoantibodies; the vinculin autoantibodies may also play an important role in the pathogenesis of gastrointestinal dysfunction in scleroderma (Brittany L. Adler, Zsuzsanna McMahan. Anti-vinculin autoantibodies in systemic sclerosis: a step toward a novel biomarker? Clinical Rheumatology, (2021) 40: 809-811.). In addition, vinculin antibodies have also been studied in irritable bowel syndrome. Chaoling Luo et al. disclosed a vinculin antibody detection kit in irritable bowel syndrome (Chaoling Luo, Xiao Chen, Min Dong. A detection kit for a vinculin antibody as an irritable bowel syndrome marker and a preparation method thereof. Application number: 201610325264.3).

However, the expression of vinculin and the existence of vinculin autoantibodies have not been reported in nephrotic syndromes. In addition, in the prior art, target-based vinculin or autoantibodies are not used as a serological marker in autoimmune nephrotic syndrome. There is no research on identifying autoimmune nephrotic syndrome by detecting an anti-vinculin-IgG antibody in serum. Compared with the detection kit for a vinculin antibody as an irritable bowel syndrome marker disclosed by Chaoling Luo et al., the kit of the present disclosure can qualitatively and quantitatively detect the anti-vinculin-IgG antibody in the serum of patients with autoimmune nephrotic syndrome. Moreover, the kit uses a human anti-tag peptide IgG antibody as a standard, combined with a biotin-avidin amplification system and magnetic particle-based chemiluminescence immunoassay, to significantly improve the detection accuracy, sensitivity, specificity, and detection speed.

### SUMMARY

The present disclosure aims to provide a kit for detecting an anti-vinculin-IgG antibody, a detection kit of target-based vinculin, or corresponding autoantibodies. The kit can detect autoantibodies from tissues (kidney biopsy) or body fluids (especially blood, plasma, and serum) by immunoreaction with the antigen protein vinculin (especially according to SEQ ID NO: 1).

The present disclosure provides a kit for detecting an anti-vinculin-immunoglobulin G (IgG) antibody, including the antigen protein vinculin, a solid phase carrier, a labeled antibody selected from the group consisting of an enzyme-labeled secondary antibody, a chemiluminescent agent-labeled secondary antibody, and a biotin-labeled secondary antibody, an antigen diluent, a sample dilution buffer, an antibody diluent, a substrate color development reagent, a washing solution, a standard, a positive quality control, and negative quality control.

The antigen protein vinculin has a sequence shown in SEQ ID NO: 1, as follows:

PKFREAVKAASDELSKTISPMVMDAKAVAGNISDPGLQKSFLDSGYRILG
AVAKVREAFQPQEPDFPPPPQLRLTDELAPPKPPLPEGEVPPPRPPPPEE
KDEEFPEQKAGEVINQPMMMAARQLHDEARKWSSKGNDIIAAAKRMALLM
AEMSRLVRGGSGTKRALIQCAKDIAKASDEVTRLAKEVAKQCTDKRLLQV
CERIPTISTQLKILSTVKATMLGRTNISDEESEQATEMLVHNAQNLMQSV
KETVREAEAASIKIRTDAGFTLRWVRKTPWYQ.

In the present disclosure, the antigen protein vinculin can be a fusion protein, using tags with certain biological or physical functions, especially an N-terminal or a C-terminal. These tags facilitate purification, immobilization, and precipitation of the antigen protein. In a preferred example, the tag may be a sequence or domain capable of specifically binding a ligand; for example, the tag peptide may be selected from the group consisting of a His tag, thioredoxin, a GST tag, a maltose-binding protein, an SA tag of glutathione S-transferase, a c-Myc tag, a Flag tag, and a biotin tag.

In the present disclosure, the antigen protein vinculin is immobilized on the solid phase carrier; preferably, the solid phase carrier may be selected from the group consisting of a nitrocellulose membrane (NC), magnetic particles, and an enzyme-labeled microplate.

For example, the standard and the positive quality control each may be selected from the group consisting of a recombinant human anti-tag peptide IgG or a fragment thereof and an anti-vinculin-IgG antibody extracted from the serum of a patient; and the negative quality control may be the serum of healthy control.

In the present study, the antigen protein vinculin may be expressed in bacteria such as Escherichia coli, saccharomycetes, and mammalian cells.

In the present study, the antigen protein vinculin may be purified by Ni column affinity chromatography, molecular sieve chromatography, ion exchange column chromatography, and hydrophobic interaction chromatography.

In the present disclosure, the biological sample may be an autoantibody-containing sample selected from the group consisting of whole blood, serum, plasma, urine, lymph, hydrothorax, and ascites, preferably mammalian (human) serum.

The kit includes a substrate color development reagent, an antigen diluent, a sample dilution buffer, an antibody diluent, and a washing solution. The substrate color development reagent may be selected from the group consisting of tetramethylbenzidine (TMB), hydrogen peroxide, 3-(2′-Spiroadamantane)-4-methoxy-4-(3″-phosphoryloxy) phenyl-1,2-dioxetane (AMPPD), 4-methylumbelliferyl phosphate (4-MUP), and 5-bromo-4-chloro-3-indolyl phosphate (BCIP); the antigen diluent may be 1× PBS at pH 7.4 containing 163 mM NaCl and 1% Triton X-100; the sample dilution buffer may be 0.01 M PBS at pH 7.4 containing 10% bovine serum albumin (BSA); the antibody diluent may be 0.01 M PBS at pH 7.4 containing 1 M D-glucose, 2% glycerol, and 0.35% Tween 20, and the washing solution may be 1× PBS at pH 7.4 containing 163 mM NaCl, 10% glycerol, and 1% Triton X-100.

In a preferred example, “immobilization” as described herein refers to binding the antigen protein vinculin to a water-insoluble solid phase carrier or support, preferably by covalent bonding, electrostatic interaction, hydrophobic interaction, or disulfide bond interaction, and preferably by one or more covalent bonds. The immobilization may be conducted by direct immobilization; for example, immobilized molecules are separated from an aqueous solution with the insoluble carrier by filtration, centrifugation, or chromatography. The antigen protein vinculin can be reversibly or irreversibly immobilized. For example, the antigen protein is immobilized on the carrier by cleavable covalent bonds (such as disulfide bonds that can be cleaved by adding thiol-containing reagents), and this immobilization is reversible. Alternatively, if the antigen protein is immobilized to the carrier via a covalent bond that does not cleave in an aqueous solution (a bond formed by a reaction of an epoxide group with an amine group that couples a lysine side chain to an affinity column), the immobilization is irreversible. Immobilization can also be conducted indirectly, such as immobilizing an antibody with a specific affinity for the antigen protein and forming an antigen protein-antibody complex to achieve immobilization.

In the present disclosure, the antigen protein vinculin is immobilized by a direct enveloping method: (1) the antigen protein vinculin is bound to the nitrocellulose membrane or the polystyrene microplate by physical adsorption or a noncovalent bond; (2) magnetic particles with carboxyl functional groups are bound to amino groups of the antigen protein vinculin, and the antigen protein vinculin is bound to the magnetic particles by chemical coupling.

In the present disclosure, the selected labeled antibody may be a horseradish peroxidase (HRP)-labeled anti-human IgG antibody, an acridinium ester-labeled anti-human IgG antibody, and a biotin-labeled anti-human IgG antibody.

In the present study, a recombinant protein, vinculin, was successfully expressed and purified by gene recombination and prokaryotic expression. The recombinant protein is used as an antigen protein in the kit to develop a kit suitable for detecting an anti-vinculin-IgG antibody in the serum of a patient with autoimmune nephrotic syndrome. The kit includes a qualitative or quantitative detection kit for detecting the anti-vinculin-IgG antibody in human serum.

A kit for detecting the anti-vinculin-IgG antibody in serum is based on indirect reaction. A vinculin antigen is adsorbed on the solid phase carrier as a coating antigen, the positive quality control or standard or a serum sample to be tested is added for incubation, and the labeled secondary antibody is added for reaction; if the serum to be tested contains anti-vinculin-IgG antibody, a ternary complex of coating antigen Vinculin-anti-Vinculin-IgG antibody of serum to be tested-labeled anti-human IgG antibody is formed. The photochromogenic chemiluminescence detects light signals and fluorescence radiation to analyze the anti-Vinculin-IgG antibody qualitatively or quantitatively in human serum.

In the present disclosure, the kit detected an anti-Vinculin-IgG autoantibody in the body of some patients with autoimmune nephrotic syndrome for the first time, and it was determined that a target antigen of the autoantibody is vinculin on podocytes. Therefore, the kit can be used for detecting the anti-Vinculin-IgG autoantibody, providing a basis for the study of the molecular mechanism and clinical diagnosis and treatment of the autoimmune nephrotic syndrome.

Compared with the prior art, the present disclosure has the following characteristics of innovation:
(1) At present, studies on vinculin and anti-vinculin-IgG antibodies in patients with renal disease at home and abroad are limited to the study of molecular mechanisms, and there is no quantitative detection of serum levels for these two substances in patients. In the present disclosure, the autoantibody against vinculin is identified for the first time, and a detection kit is prepared for the anti-Vinculin-IgG autoantibody, thus filling the gap in related fields at home and abroad. The anti-Vinculin-IgG antibody in the serum of 466 patients with nephrotic syndrome was detected using the kit, and the results showed that 262 patients were positive for the anti-Vinculin-IgG antibody, indicating that the anti-Vinculin-IgG antibody had a positive detection rate of 56.2%.
(2) In the present disclosure, the kit involves the qualitative analysis of anti-Vinculin-IgG antibodies in human serum by solid-phase membrane immunoassay, and the human anti-tag peptide IgG antibody is used as a standard, which greatly improves the detection accuracy. The solid-phase membrane immunoassay is simple in operation, with less reagent consumption, nearly ten times lower than traditional ELISA; in addition, the NC membrane has an extremely strong adsorption capacity of close to 100%, such that trace antigen can be completely absorbed and immobilized on the NC membrane; the NC membrane adsorbed with antigen or antibody or with existing results can be stored for a long time (at −20° C. for half a year) without affecting the activity. In addition, the kit for the qualitative detection of the anti-Vinculin-IgG antibody in human serum by the solid-phase membrane immunoassay introduces a biotin-avidin amplification system, thereby significantly improving the detection sensitivity.
(3) In the present disclosure, a kit for quantitatively detecting the anti-Vinculin-IgG antibody in human serum is related to magnetic particle-based chemiluminescence immunoassay, which uses magnetic particles as a solid phase carrier, with a diameter of only 1.0 μm. This dramatically increases the coating surface area, increases the amount of antigen adsorbed, improves the reaction rate, and makes cleaning and separation easier, thereby reducing contamination and the probability of cross-infection. On the other hand, the anti-human IgG is directly labeled by an acridinium ester luminescent agent with a simple, rapid, and catalyst-free chemical reaction; the acridinium ester has flash-type chemiluminescence and can achieve a maximum emission intensity after 0.4 s by starting a luminescent reagent (H₂O₂, NaOH); the acridinium ester has a half-life of 0.9 s, which can be terminated within 2 s, thereby realizing rapid detection.

### BRIEF DESCRIPTION OF THE DRAWINGS

FIG. 1 shows that vinculin on podocytes is the main target antigen for autoantibodies in a patient with autoimmune nephrotic syndrome; FIG. 1A: a primary antibody is a two-dimensional electrophoresis protein spot of healthy human serum; FIG. 1B: a primary antibody is a two-dimensional electrophoresis protein spot in the serum of a patient with autoimmune nephrotic syndrome; FIG. 1C: mass spectrometry identification of the target antigen Vinculin;

FIG. 2 shows SDS-PAGE identification of the expressed recombinant protein vinculin;

FIG. 3 shows the detection of an anti-Vinculin-IgG antibody in the serum of a patient with autoimmune nephrotic syndrome by a solid-phase membrane immunoassay kit.

FIG. 4 shows a schematic diagram for the detection of the anti-Vinculin-IgG antibody by a magnetic particle-based chemiluminescence immunoassay kit.

FIG. 5 shows a schematic diagram of the antigen protein vinculin coated with carboxyl magnetic particles.

FIG. 6 shows the detection of the anti-Vinculin-IgG antibody in patients with various types of renal diseases, where NS: autoimmune nephrotic syndrome, HSP: Henoch-schonlein purpura, HSPN: Henoch-schonlein purpura nephritis, IgAN: IgA nephropathy, and NC: healthy children.

FIG. 77 shows a ROC curve to evaluate the use value of the anti-Vinculin-IgG antibody as a serological marker for diagnosing patients with autoimmune nephrotic syndrome.

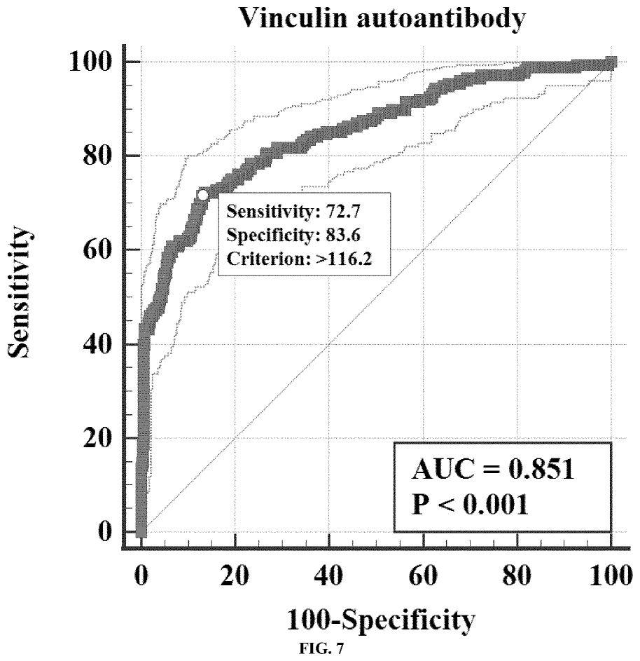

### DETAILED DESCRIPTION OF THE EMBODIMENTS

The present disclosure will be described in further detail below with reference to the accompanying drawings and specific embodiments. The following embodiments are only intended to illustrate the disclosure rather than limiting the scope of the disclosure.

### EXAMPLE 1 VINCULIN ON PODOCYTES AS THE MAIN TARGET ANTIGEN FOR AUTOANTIBODIES IN A PATIENT WITH AUTOIMMUNE NEPHROTIC SYNDROME

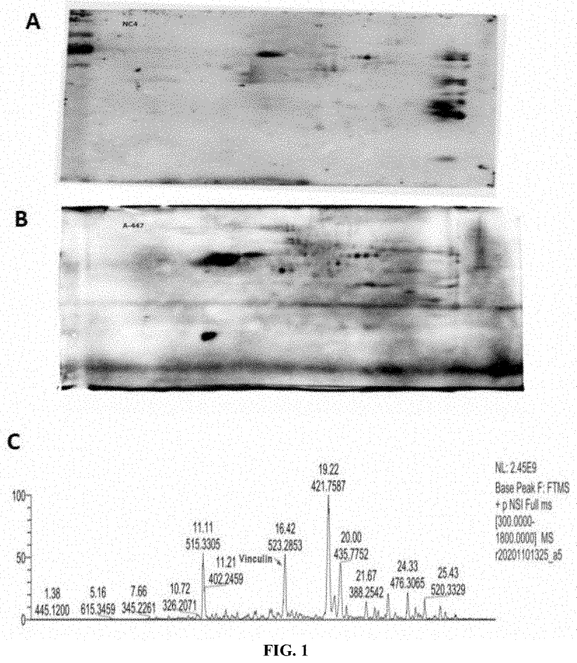

In the present disclosure, through many clinical and molecular mechanism studies in an early stage, it was found for the first time that patients with nephrotic syndrome have a higher serum IgG level. Furthermore, it was confirmed that vinculin on podocytes was the target antigen for autoantibodies in patients with autoimmune nephrotic syndrome. Therefore, detecting the presence and quantitative levels of the anti-Vinculin-IgG antibody in serum was helpful for the early identification of autoimmune nephrotic syndrome, especially for screening patients with related symptoms. The specific implementation included the following: (1) extraction of total protein from glomerular podocytes: a sample of a podocyte line (MPCS) was cultured, washed 2 to 3 times with PBS, and subjected to extensive lysis on ice using a focused sonicator (Covaris S220, Gene) in lysis buffer containing 30 mM Tris-HCl, 8 M urea, 4% CHAPS and a protease inhibitor (#ab65621; Abcam, 1:200 dilution), and the sample was centrifuged at 12,000 g and 4° C. for 30 min. The supernatant was collected to obtain the total protein of glomerular podocytes. The total protein concentration of the glomerular podocytes was determined using a BCA protein concentration assay kit. (2) Two-dimensional electrophoresis: the total protein of glomerular podocytes was subjected to two-dimensional electrophoresis and transferred to a nitrocellulose membrane; sera of healthy people and patients with autoimmune nephrotic syndrome were used as primary antibodies for incubation separately, and a secondary antibody was added for development, as shown in FIG. 1A and FIG. 1B. (3) Matrix-assisted laser desorption ionization time-of-flight mass spectrometry (MALDI-TOF-MS): after development in step (2), differential analysis was conducted on positive spots; protein spots were strongly positive in nephrotic syndrome patients, and negative or weakly positive in healthy people on the two-dimensional electrophoresis gel were selected, and the selected protein spots were cut out from the gel; a dried gel was digested with trypsin (0.1 μg/μl ), 10 μl of 25 mM ammonium bicarbonate was added to a reaction mixture, incubated overnight at 37° C., and peptides were extracted from the gel with trifluoroacetic acid (0.1%). The extracted peptide was analyzed by a MALDI-TOF-MS mass spectrometer to obtain a mass spectrum of the peptide, which was identified as Vinculin protein, as shown in FIG. 1C.

### EXAMPLE 2 EXPRESSION AND PURIFICATION OF THE RECOMBINANT ANTIGEN PROTEIN VINCULIN

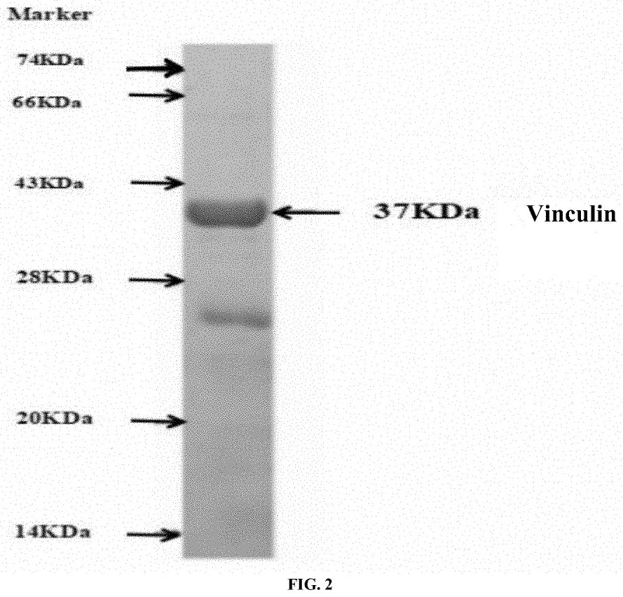

Genetic engineering used a gene encoding the vinculin protein as a template for PCR amplification, and an expression carrier was constructed for protein expression. Tag peptides containing His tags were on expressed antigen proteins. The expressed recombinant protein was purified by nickel column affinity chromatography, ion affinity chromatography, hydrophobic interaction chromatography, and molecular sieve, and the molecular weight of the recombinant protein vinculin was identified by SDS-PAGE as 37 kDa, as shown in FIG. 2.

### EXAMPLE 3 OPTIMIZATION OF THE REACTION CONDITIONS OF A KIT BY ORTHOGONAL EXPERIMENTAL DESIGN

According to a coating concentration of the antigen Vinculin (50 μg/ml, 80 μg/ml, 100 μg/ml, and 150 μg/ml), each reaction time (15 min, 30 min, and 45 min), temperature (25° C. and 37° C.), and an optimal dilution of enzyme-labeled secondary antibody (1:100, 1:500, 1:1000, 1:1500), an orthogonal table was selected, each factor was repeated at two levels to determine standard positive serum and standard negative serum, and a ratio (P/N) of the highest light signal value (P) of the positive serum and the lowest light signal value (N) of the negative serum was selected. By the orthogonal design, the kit had an optimal antigen Vinculin coating concentration of 80 μg/ml, the solid-phase membrane immunoassay for the anti-Vinculin-IgG antibody kit had an optimal antigen-antibody reaction temperature of 25° C., an optimal antigen-antibody reaction time of 30 min, and an optimal working dilution of the optimal biotin-labeled anti-human IgG antibody at 1:500; the magnetic particle-based chemiluminescence immunoassay for anti-Vinculin-IgG antibody kit had an optimal antigen-antibody reaction temperature of 37° C., an optimal antigen-antibody reaction time of 15 min, and an optimal working dilution of the optimal acridinium ester-labeled anti-human IgG antibody at 1:500.

### EXAMPLE 4 PREPARATION OF A SOLID-PHASE MEMBRANE IMMUNOASSAY KIT FOR DETECTING AN ANTI-VINCULIN-IGG ANTIBODY

4.1. Composition of a solid-phase membrane immunoassay kit for detection of the anti-Vinculin-IgG antibody:
1. Antigen: recombinant protein vinculin
2. Solid phase carrier: Satourius CN140 nitrocellulose membrane
3. Positive quality control (standard): human anti-His tag IgG (purchased from Huzhou Yingchuang)
4. Negative quality control: serum of healthy controls
5. Labeled antibody: biotin-labeled anti-human IgG antibody
6. Antigen diluent
7. Sample dilution buffer
8. Antibody diluent
9. Washing solution
10. Enzyme working solution: alkaline phosphatase-streptavidin
11. Substrate color development reagent: BCIP color development reagent.

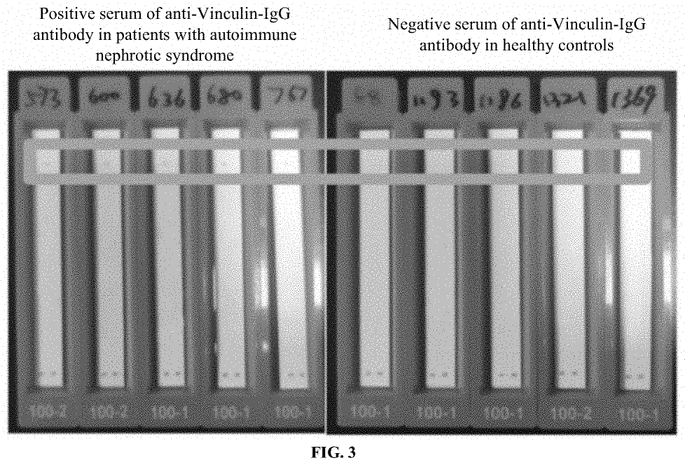

4.2. The detection steps of the solid-phase membrane immunoassay kit for detection of the anti-Vinculin-IgG antibody included the following:
4.2.1. Coating and blocking: 8 μl of Vinculin antigen with a concentration of 80 μg/ml was added dropwise directly to the nitrocellulose membrane and dried in a 37° C. incubator for 30 min, and the nitrocellulose membrane was blocked on a detection plate with 200 ul of 5% BSA in a 37° C. incubator for 30 min. After discarding the blocking solution, the nitrocellulose membrane was washed twice with a washing solution.
4.2.2. Antigen incubation: 10 μl of an antibody standard or serum to be tested diluted with diluent was added to the detection plate, while negative and positive controls were set up at the same time and then incubated at 25° C. for 30 min, where three parallel wells were set for each sample.
4.2.3. Secondary antibody incubation: The liquid in the detection plate was discarded, the plate was washed five times with a washing solution for 1 min each time, and 20 μl of a 1:500 biotin-labeled anti-human IgG antibody was added and incubated at 25° C. for 30 min.
4.2.4 Color development: The liquid in the detection plate was discarded, the plate was washed five times with the washing solution for 1 min each time, and 500 μl of alkaline phosphatase-streptavidin was added and incubated at room temperature for 20 min. The liquid in the detection plate was discarded, and the plate was washed five times with the washing solution for 1 min each time. A color development reagent BCIP was added and reacted at room temperature for 20 min, and the detection plate was rinsed with running water to stop the enzyme reaction. The nitrocellulose membrane test strip was removed and dried with a hairdryer and qualitatively determined with a colorimetric card by the naked eye. Those with prominent brown spots were positive, as shown in FIG. 3. Alternatively, the membrane strip was scanned on a developing device. Analysis software that comes with the developing device used the reference standard concentration as ordinate and the gray value read by the device as an abscissa to draw a standard curve for the semiquantitative analysis of anti-Vinculin-IgG antibody levels in serum.

### EXAMPLE 5 PREPARATION OF A MAGNETIC PARTICLE-BASED CHEMILUMINESCENCE IMMUNOASSAY KIT FOR DETECTING AN ANTI-VINCULIN-IGG ANTIBODY

5.1. Composition of a magnetic particle-based chemiluminescence immunoassay kit for detection of the anti-Vinculin-IgG antibody:
1. Antigen: recombinant protein vinculin
2. Solid phase carrier: magnetic particles with carboxyl functional groups
3. Positive quality control (standard): human anti-His tag IgG (purchased from Huzhou Yingchuang)
4. Negative quality control: serum of healthy controls
5. Labeled antibody: acridinium ester-labeled anti-human IgG antibody
6. Antigen diluent
7. Sample dilution buffer
8. Antibody diluent
9. Washing solution
10. Preexcitation solution: H₂O₂
11. Excitation solution: NaOH.

5.2. Principle of a magnetic particle-based chemiluminescence immunoassay kit for detection of the anti-Vinculin-IgG antibody

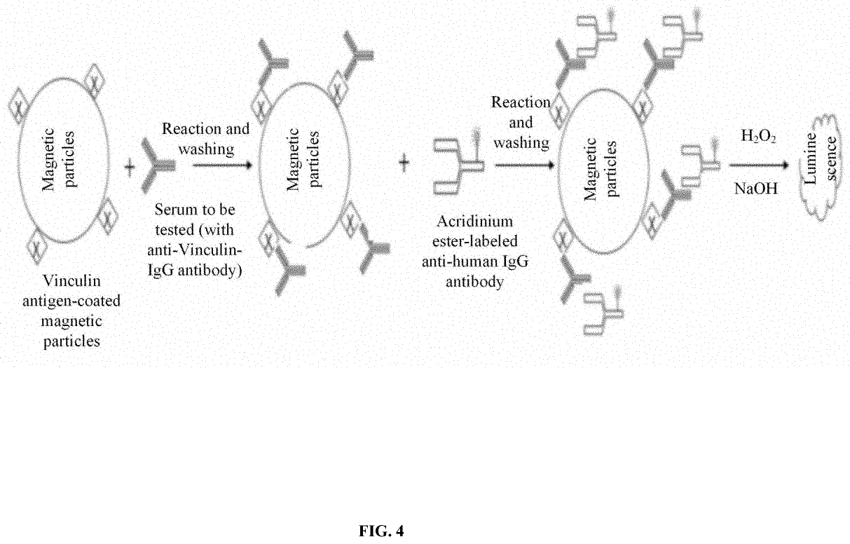

The chemiluminescence immunoassay kit is an analytical method combining magnetic separation, immunoassay, and chemiluminescence. The kit used an indirect method to quantitatively detect anti-Vinculin-IgG antibody in human serum: a Vinculin antigen-coated magnetic particle solution was mixed with a diluted sample, and specific anti-Vinculin-IgG antibodies were bound to the Vinculin antigen-coated magnetic particles; after washing, an acridinium ester-labeled anti-human IgG antibody was added to form a Vinculin antigen-coated magnetic particle-anti-Vinculin-IgG antibody-acridinium ester-labeled anti-human IgG antibody complex; under the action of an external magnetic field, the unbound substance and the complex formed by the immune reaction were separated, a supernatant was discarded, a precipitated complex was washed, and the preexcitation solution (H₂O₂) and the excitation solution (NaOH) were added to conduct a luminescence reaction; under alkaline conditions, the acridinium ester molecule was attacked by the hydrogen peroxide to generate dioxyethane, which was unstable and decomposed into CO₂ and N-methylacridone in an electronically excited state, returning to the ground state, N-methylacridone emitted light with a wavelength of 430 nm, and a luminescence intensity was determined using a chemiluminometer. The concentration of anti-Vinculin-IgG antibody was proportional to the luminescence intensity, and the concentration of anti-Vinculin-IgG antibody in the serum to be tested was calculated by a calibration curve, as shown in FIG. 4.

5.3. Preparation of vinculin antigen-coated magnetic particles

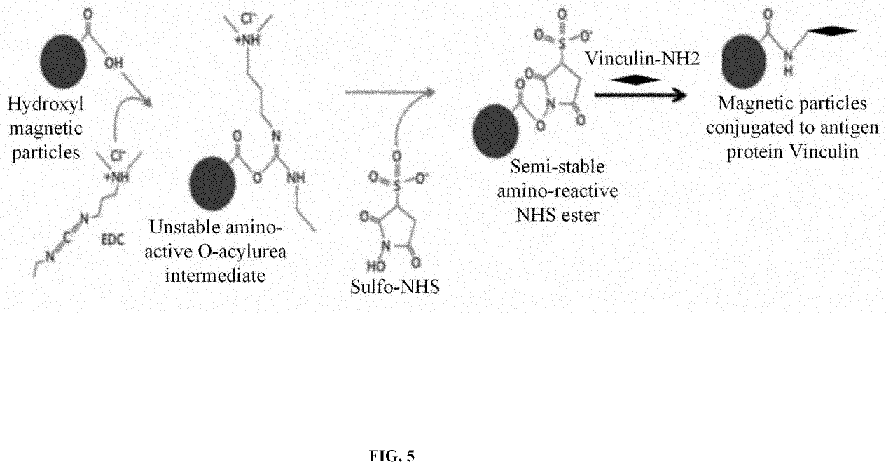

5.3.1 The principle of coating magnetic particles with vinculin antigen: the carboxyl functional groups contained on the surface of the magnetic particles reacted with an EDC (1-(3-dimethylaminopropyl)-3-ethylcarbodiimide) solution to generate an unstable amino-active O-acylurea intermediate; the intermediate reacted with NHS (N-hydroxysuccinimide) to form a semistable amino-reactive NHS ester; the semistable amino-reactive NHS ester reacted with amino groups on the antigen protein vinculin to form the vinculin antigen-coated magnetic particles, as shown in FIG. 5.

5.3.2. For the EDC/NHS activation of the carboxyl magnetic particles, the specific steps were as follows:
a) A total of 10 mg of magnetic particles was washed three times with 20 mM MES and separated by a magnet, and the supernatant was discarded.
b) The washed magnetic particles were resuspended in 100 μl of 20 mM MES to a final 100 mg/ml concentration.
c) Fifty microliters of 20 mg/ml EDC and 50 μl of 24 mg/ml Sulfo-NHS that were prepared in PBS were added to the cleaned magnetic particles in sequence, mixed well, and allowed to stand at room temperature for activation for 30 min.
d) After an external magnetic field action, the supernatant was discarded, and the magnetic particles were washed with 400 μl of 0.05 M PBS, diluted with 400 μl of a preservation solution, and stored for later use.

5.3.3 Crosslinking of activated magnetic particles with antigen protein Vinculin: precooled 1 ml of 20 mM MES was added to wash the activated magnetic particle solution twice; 200 μl of the 2 mg/ml antigen protein vinculin was added to the activated magnetic particles, mixed thoroughly, and allowed to stand for reaction at room temperature for 16 h; after the reaction, PBS containing 0.2% Tween 20 at pH 7.4 was added, and the magnetic particles were washed twice; PBS containing 0.2% Tween 20 and 0.2% BSA at pH 7.4 was added until a final concentration of the magnetic particles was 10 mg/ml, mixed well, and allowed to stand for reaction at room temperature for 30 min; after the reaction, the supernatant was discarded, and the magnetic particles were resuspended in PBS containing 0.2% Tween 20, 0.2% BSA at pH 7.4, such that the cross-linking of the activated magnetic particles and the antigen protein vinculin was completed.

5.4. To prepare the acridinium ester-labeled anti-human IgG antibody, the specific steps were as follows:
a) A 2 mg/mL acridinium ester solution was prepared by dimethylformamide.
b) A 1 mg/mL anti-human IgG antibody was prepared by a 0.2 M (pH 8.0) carbonate buffer.
c) The acridinium ester and the anti-human IgG antibody was thoroughly mixed at a molar ratio of 4:1 and reacted for 40 min.
d) The reaction was terminated with 20 μl of carbonate buffer containing 5% lysine.
e) desalting and impurity removal were conducted to obtain an acridinium ester-labeled anti-human IgG antibody solution with higher purity.

5.5. The steps of detecting anti-Vinculin-IgG antibody in serum by a magnetic particle-based chemiluminescence immunoassay kit were as follows:

5.5.1 Serum to be tested: 100 μl of a diluted serum or an anti-His-tag IgG standard was added to 100 μl of the magnetic particle solution coated with antigen protein Vinculin and reacted at 37° C. for 15 min. At the same time, negative and positive controls were set up.

5.5.2 Labeled antibody: A total of 400 μl of a washing solution was added to wash three times for 1 min each time, and 100 μl of an acridinium ester-labeled anti-human IgG antibody diluted by 1:500 was added and reacted at 37° C. for 15 min.

5.5.3 Signal detection: precipitated complexes were washed with 400 μl of the washing solution three times for 1 min each time, and 100 μl of a preexcitation solution (H₂O₂) and 100 μl of an excitation solution (NaOH) were added for the reaction. A chemiluminometer detected the luminescence signal, and the luminescence value was recorded. The concentration of anti-vinculin-IgG antibody was proportional to the luminescence intensity, and a standard curve calculated the concentration of anti-vinculin-IgG antibody in the serum to be tested.

### EXAMPLE 6 CLINICAL USE OF A KIT FOR DETECTING AN ANTI-VINCULIN-IGG ANTIBODY IN SERUM

6.1 Subjects included patients diagnosed with various types of nephropathies from June 2018 to June 2020, including 466 cases of NS, 168 cases of HSP, 137 cases of HSPN, 133 cases of IgAN, and 195 cases of NC during the same period. Serum samples were obtained from various nephropathy patients and healthy controls. All subjects had their first serum sample collection before immunosuppressive therapy.

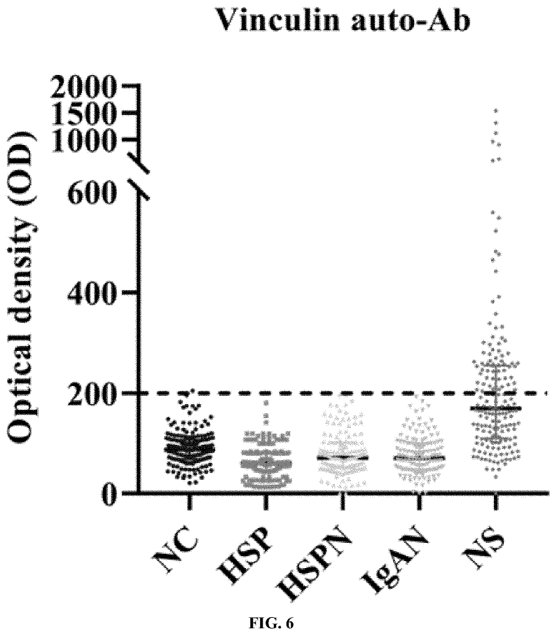

6.2 Detection of anti-Vinculin-IgG antibodies in patients with various types of nephropathies: The kit for the present disclosure was used to detect anti-Vinculin-IgG antibody levels in the serum of patients diagnosed with various types of nephropathies from June 2018 to June 2020, including 466 cases of NS, 168 cases of HSP, 137 cases of HSPN, 133 cases of IgAN, and 195 cases of NC during the same period. The results showed positive anti-Vinculin-IgG antibodies in patients with autoimmune nephrotic syndrome and negative anti-Vinculin-IgG antibodies in patients with HSPN, HSP, IgAN, and NC, as shown in FIG. 6.

6.3 ROC curve: The value of anti-Vinculin-IgG antibody was evaluated as a serological marker in the diagnosis of patients with autoimmune nephrotic syndrome; detection results of anti-Vinculin-IgG antibody in patients with autoimmune nephrotic syndrome in 6.2 were analyzed using the ROC curve to evaluate the use value of the anti-Vinculin-IgG antibody in the diagnosis of autoimmune nephrotic syndrome. The results showed that the anti-Vinculin-IgG antibody was a desirable serological marker for the diagnosis of patients with autoimmune nephrotic syndrome; the anti-Vinculin-IgG antibody (with a cutoff value greater than 116.2 as a criterion) as a serological marker for the diagnosis of the autoimmune nephrotic syndrome had a sensitivity of 72.7%, a specificity of 83.6% and an area under the curve of 0.851, as shown in FIG. 7.

1. Use of an antigen protein vinculin in preparation of a kit for detecting autoimmune nephrotic syndrome, wherein the kit comprises an antigen protein vinculin, a solid phase carrier, a labeled antibody, an antigen diluent, a sample dilution buffer, an antibody diluent, a substrate color development reagent, a washing solution, a standard, a positive quality control, and a negative quality control; the antigen protein vinculin has a sequence shown in SEQ ID NO: 1; the labeled antibody is selected from the group consisting of an enzyme-labeled secondary antibody, a chemiluminescent agent-labeled secondary antibody, and a biotin-labeled secondary antibody; the antigen protein vinculin has a tag peptide; the standard and the positive quality control each are selected from the group consisting of a recombinant human anti-tag peptide immunoglobulin G (IgG) and an anti- vinculin-lqG antibody extracted from serum, and the negative quality control is the serum of a healthy control; and the autoimmune nephrotic syndrome is diagnosed by detecting the anti-vinculin-lqG antibody in a serum sample of a patient.

2. (canceled)

3. (canceled)

4. The use according to claim 1, wherein the tag peptide is selected from the group consisting of a His tag, thioredoxin, a GST tag, a maltose-binding protein, an SA tag of glutathione S-transferase, a c-Myc tag, a Flag tag, and a biotin tag.

5. The use according to claim 1, wherein the antigen protein vinculin is purified by nickel column affinity chromatography, molecular sieve chromatography, gel filtration chromatography, ion exchange column chromatography, or hydrophobic interaction chromatography.

6. (canceled)

7. The use according to claim 1, wherein the antigen protein vinculin is immobilized on the solid phase carrier selected from the group consisting of a nylon membrane, an ion exchange resin, a nitrocellulose membrane, a polystyrene microplate, and magnetic particles.

8. The use according to claim 7, wherein the antigen protein vinculin is directly immobilized on the solid phase carrier by physical adsorption, covalent bonding, or chemical bonding.

9. The [[kit]] use according to claim 1, wherein the substrate color development reagent is selected from the group consisting of tetramethylbenzidine (TMB), hydrogen peroxide, 3-(2′-Spiroadamantane)-4-methoxy-4-(3″-phosphoryloxy)phenyl-1,2-dioxetane (AMPPD), 4-methylumbelliferyl phosphate (4-MUP), and 5-bromo-4-chloro-3-indolyl phosphate (BCIP); the antigen diluent is a 1× PBS at a pH value of 7.4 containing 163 mM NaCl and 1% Triton X-100; the sample dilution buffer is a 0.01 M PBS at a pH value of 7.4 containing 10% bovine serum albumin (BSA); the antibody diluent is the 0.01 M PBS at a pH value of 7.4 containing 1M D-glucose, 2% glycerol, and 0.35% Tween 20; and the washing solution is the 1× PBS at a pH value of 7.4 containing the 163 mM NaCl, 10% glycerol, and the 1% Triton X-100.
